(19) United States
(12) Patent Application Publication
(10) Pub. No.: US 2025/0009250 A1
(43) Pub. Date: Jan. 9, 2025

(54) DIAGNOSTIC, CONTROL AND THERAPY SYSTEM FOR IDENTIFYING AND TREATING RESPIRATORY EVENTS OCCURRING WHILST SLEEPING

(71) Applicant: DIAMETOS GMBH, Potsdam (DE)

(72) Inventor: Christoph JANOTT, Berlin (DE)

(21) Appl. No.: 18/710,636

(22) PCT Filed: Nov. 8, 2022
(86) PCT No.: PCT/EP2022/081100

§ 371 Date: May 16, 2024

(30) Foreign Application Priority Data
Nov. 16, 2021 (DE) 10 2021 129 912.8

(51) Int. Cl. A61B 5/08; A61B 5/00

(52) U.S. Cl. CPC A61B 5/0803; A61B 5/4818; A61B 5/4836; A61B 5/7267

(57) ABSTRACT

The present invention relates to a diagnostic and control system (10) for continuously identifying respiratory events and their respective causal obstruction types (O1-O4) for airways of a sleeping patient and for a counteracting control of a therapy apparatus (6), comprising:
a) a first detector (1) which receives at least one first patient signal and identifies exceeding of the upper or lower limit of a predetermined tolerance range by the first patient signal and generates a respiratory event signal (1b) on the basis thereof,
b) a second detector (2) which receives the first and/or at least one second patient signal and processes this as a classifier in order to identify a first obstruction type (O1-O4) therefrom, and which outputs a corresponding obstruction type signal (2b); and
c) a control unit (4) which processes the event signal (1b) and the obstruction type signal (2b) and which generates a control signal (4b) for controlling the therapy apparatus (6) if the event signal (1b) is positive and the obstruction type signal (2b) corresponds to the first obstruction type (O1-O4).

The present invention relates to a diagnostic and control system for continuous detection of respiratory events and their respective causative types of obstruction of the airway of a sleeping patient and for counteracting control of a therapy apparatus.

Patients who suffer from upper airway obstructions that occur during sleep usually produce snoring noises during sleep, which can disturb other people. However, this also often results in damage to the organism caused by a nocturnal drop in blood oxygen saturation and a disturbance of the natural sleep structure. Partial (hypopnoea) or complete (apnoea) obstruction of the upper airway leads to a respiratory event in which the respiratory circulation deviates from a normal state without the obstruction. The respiratory event then leads to a temporary drop in blood oxygen saturation (SpO2), which can be measured using a pulse oximeter, for example. In many cases, the respiratory event can also be determined by an acoustic measurement of breathing sounds or snoring noises, but only qualitatively. In order to resolve the respiratory event or the obstruction, solutions are known which provide special stimulation of the patient when a respiratory event is detected.

WO 2011 077 433 A1 discloses a diagnostic and control system that measures a tongue position of the patient and, depending on the tongue position, infers a respiratory event and thereby triggers an action in close temporal relation in order to counteract the respiratory event.

US 2019/0160282 A1 discloses a diagnostic and control system that detects physiological parameters that indicate apnoea, such as thoracic movements, and, depending on information from the diagnostic system, sends a stimulation signal to the patient, for example to subcutaneously placed stimulation electrodes, in order to counteract the respiratory event.

WO 2013 046 049 A2 discloses systems and methods for stimulating nerves or muscles, wherein a stimulation intensity, such as a hypoglossal nerve stimulation, is generated depending on a severity of the respiratory event.

In principle, the number and strength of stimulations should be kept to a minimum, as any stimulation can disturb sleep and also consumes energy, which is limited, especially in battery-operated systems.

With some known systems, only a tongue position is evaluated, where an anatomical cause of the respiratory event can also be caused by other anatomical structures in addition to the tongue, such as palatine tonsils, a soft palate, a circular narrowing of the pharyngeal walls or an epiglottis. Because the condition of these other anatomical structures is not evaluated, respiratory events may be missed.

In some known diagnostic and control systems, only a limited therapeutic effect is achieved by a predetermined type of stimulation as stimulation of a predetermined anatomical site, such as tongue stimulation, an active mandibular prognathism splint, or implanted magnets. If the detection of the respiratory event in an apnoea occurs without consideration of an anatomical cause, such as a measurement of thoracic movement, unnecessary stimulation may be triggered, such as tongue stimulation, but if the apnoea is caused by, for example, a circular constriction of the pharyngeal walls, tongue stimulation is not effective against the same.

It may be desirable to differentiate between non-obstructive snoring, hypopnoea and apnoea, for example to trigger an action only when apnoea occurs and not when non-obstructive snoring occurs. This saves energy and disturbs sleep as little as possible.

DE 1020 19 105 762 A1 discloses a system for detecting different types of obstruction based on an acoustic snoring sound signal in the case of sleep apnoea and a corresponding method for doing so. The system comprises an input interface for the respective snoring sound signal, a first classifier which can be trained to recognize and output the most probable cause of snoring sound for a respective snoring sound signal, a second classifier which can be trained to recognize and output the most probable mouth position for a respective snoring sound signal, and a third classifier or a link matrix which is designed to recognize the most probable obstruction type from the snoring sound signal to be examined, the specific snoring sound origin type and the mouth position determined for the same, and to output these factors.

Snoring is the term used to describe nocturnal breathing noises caused by vibrations of soft tissue in the upper airway. There are different definitions of the term “snoring”, where snoring can be regarded as a vibration of the tissue of the upper airway caused by an airflow, which produces a snoring sound with a significant tonal component. However, a distinction between snoring and noisy breathing is not clearly defined. In the following, the term “snoring” or “snoring noise” is understood to be a general breathing noise that contains breathing noises with and/or without a significant tonal component. The snoring sound as an acoustic signal is usually recorded by a microphone or microphone array and converted into an electrical snoring sound signal. The snoring sound signal could also comprise additional information as indicators or labels or be a multi-channel signal, for example to detect a snoring sound origin and/or a snoring sound type and/or a mouth position. The snoring sound signal could also be associated with a time, a patient identifier, a patient weight and/or a sleeping position.

Obstructive sleep apnoea is a condition in which breathing stops at night due to obstructions (constrictions or occlusions) of the upper airway, known as airway obstructions. A single obstructive event can last from a few seconds to over a minute. Depending on the number of obstructive events per hour, different degrees of severity of obstructive sleep apnoea are distinguished. Snoring is a common accompanying symptom of obstructive sleep apnoea. Snoring and obstructive sleep apnoea are sleep-related breathing disorders. Obstructive sleep apnoea is in the following also briefly referred to as sleep apnoea.

The snoring noises and airway obstructions occur at different locations in the upper airway and include different types. The different types can be determined by the respective orientation and shape of the vibration or constriction, which can be circular or laterally slit-shaped, for example. Accordingly, there are different causes of snoring sounds that are anatomically related to the different obstruction locations and types. In other words, the snoring sound origin type is defined by the snoring sound origin location, the orientation and shape of the vibration, or a combination of these. Similarly, the type of obstruction is defined in the following by the location of the obstruction, the orientation of the obstruction, or a combination thereof.

The task of the invention, in order to eliminate the disadvantages of the prior art, is therefore to provide a diagnostic and control system with which respiratory events of a sleeping patient caused by airway obstruction are recognized and which controls a counteracting therapy device in the most targeted, efficient and patient-friendly manner possible.

The above task is solved by a device according to the features of independent claim 1 and by a device according to the features of independent claim 10. Further advantageous embodiments of the invention are given in the dependent claims.

According to the invention, a diagnostic and control system is provided for a continuous detection of respiratory events and their respective causative obstruction types of the airway of a sleeping patient and for a counteracting control of a therapy device, comprising the following:
a) a first detector which receives at least one first patient signal as an electrical signal via a first signal input and is designed to recognize when the first patient signal exceeds or falls below a predetermined tolerance range of signal values and to generate a respiratory event signal therefrom, wherein the predetermined tolerance range of the signal values is representative of sleeping without a respiratory event and is stored in the first detector;
b) a second detector which receives the first and/or at least one second patient signal as an electrical signal via a second signal input and processes it as a classifier so as to recognize therefrom a first obstruction type as a most probable obstruction type from a group of predetermined obstruction types and to output a corresponding obstruction type signal with the first obstruction type; and
c) a control unit which receives the event signal and the obstruction type signal and, if the event signal is positive and the obstruction type signal corresponds to the first obstruction type, generates therefrom a control signal which can be used to control the therapy apparatus.

The advantages achieved with the invention consist in particular in the fact that instead of simply generating a control signal for stimulation of the patient in response to a respiratory event signal, without taking into account a type of cause for this, the control signal for the therapy apparatus is only generated if the predetermined obstruction type is also present, which the therapy apparatus can remedy or alleviate. For example, if a hypoglossal stimulator is present as a therapy apparatus and is connected to the patient, the control signal is only generated if the obstruction is caused by an anatomical constriction at the level of the base of the tongue, but not if the obstruction is caused at the level of the soft palate, as hypoglossal stimulation would not be effective or only insufficiently effective against the obstruction in the latter case.

This has the decisive advantage for the patient that there is no unnecessary stimulation. In addition, this has the decisive advantage for the system, especially if it is an implanted, battery-operated system, that no unnecessary energy is consumed.

For clarity, the respiratory event is a respiratory flow change, which can be a respiratory impairment or a respiratory interruption, which can be caused by a partial or complete temporary obstruction of the upper airway.

The first patient signal is a signal that can detect a respiratory event therein. Preferably, the first detector is designed to statistically evaluate the first patient signal in a learning mode in such a way that an average value and a tolerance range of the signal values are determined, which is stored as the predetermined tolerance range. Preferably, the first patient signal is a dynamic pressure value signal from an air pressure sensor and preferably from an air pressure sensor positioned near the nostrils. The first detector evaluates the first patient signal in such a way that if the periodically occurring maximum value of an air pressure falls below a predetermined threshold value, the respiratory event is detected, and the event signal is generated accordingly.

Preferably, the first patient signal can be transmitted wirelessly to the diagnostic system, where all wired solutions are also conceivable. Alternatively, preferably, the mean value and the predetermined tolerance range can initially be entered by an operator and subsequently be automatically readjusted, preferably adaptively. For clarity, the signal values can be understood to be an amplitude or other signal values derived from the at least one first patient signal, such as frequency values, maximum values, percentiles, variance values, mean values or other statistical descriptions of the patient signal. The patient signal can also be pre-processed before evaluation, for example using a bandpass filter or other linear or non-linear analog or digital filters. This also applies to the second patient signal. Instead of the dynamic pressure signal, an acoustic microphone signal can also be used, where the first detector detects the respiratory event, for example, when a snoring sound with less than a predetermined amplitude or below a threshold value is detected.

Preferably, the first detector is designed as a classifier or with pattern recognition in order to determine the respiratory event signal from the at least one first patient signal. For clarity, the first patient signal can contain several individual sensor signals, with the advantage that the respiratory event signal can be determined more reliably. In general, the classifier or pattern recognition can be one of the following: a Support Vector Machine (SVM), a Naive Bayesian System, a Least Mean Square method, a k-Nearest Neighbor method (k-NN), a Linear Discriminant Analysis (LDA), a Random Forests method (RF), an Extreme Learning Machine (ELM), a Multilayer Perceptron (MLP), a Deep Neural Network (DNN), a Convolutional Neural Network (CNN), a logistic regression.

The second patient signal can also contain several individual sensor signals.

The second detector and its classifier are designed to preferably also recognize a second obstruction type or more, each of which is mapped in the obstruction type signal. Preferably, the second detector is provided with the classifier or a pattern recognition system in order to determine the obstruction type signal from the at least one second patient signal. The classifier or pattern recognition can be one of the following: Support Vector Machine (SVM), Naive Bayes System, Least Mean Square method, k-Nearest Neighbors method (k-NN), Linear Discriminant Analysis (LDA), Random Forests method (RF), Extreme Learning Machine (ELM), Multilayer Perceptron (MLP), Deep Neural Network (DNN), Convolutional Neural Network (CNN), logistic regression.

Preferably, the first patient signal is one or a combination of the following: a pulse oximeter signal, a microphone signal of a breathing and/or snoring sound, a respiratory flow signal, an air pressure signal, a chest muscle signal or a breathing belt signal, an EEG signal, an ECG signal, an EMG signal, a position sensor signal, a motion sensor signal or a motion sensor signal of a movement of an anatomical structure of the upper airway.

Particularly preferably, the first patient signal is a microphone signal of a respiratory sound which is recorded in the vicinity of the patient.

It is understood that one of the respective sensors required for the respective patient signals described above can be connected or is connected to the first and second signal inputs. The sensors can be an integrated part of the diagnostic and control system or can be connected separately, for example via a wire connection or wirelessly.

Preferably, the second patient signal is one or a combination of several of the following: the first patient signal, a microphone signal of a breathing and/or snoring sound, a respiratory flow signal, a signal of a palate sensor, a signal of a tongue position sensor, an air pressure sensor signal, a combination of several air pressure sensor signals, an EMG signal or a movement sensor signal of a movement of an anatomical structure of the upper airway. Particularly preferably, the second detector is designed to determine the obstruction type signal as described in DE 1020 19 105 762 A1.

Preferably, the diagnostic and control system comprises a third detector which is supplied with a third patient signal which is the first and/or the second and/or another third patient signal of a sensor on or near the patient, and which the third detector evaluates with respect to an amplitude or a time response such that it thereby generates a severity signal which is a function of a severity of the respiratory event, wherein the control unit receives the severity signal and processes it such that the control signal is a function of the severity signal.

Preferably, the third patient signal is one or a combination of several of the following: a pulse oximeter signal, a microphone signal of a breathing and/or snoring sound, a respiratory flow signal, a chest muscle signal or a breathing belt signal, an EEG signal, an ECG signal, an EMG signal, a position sensor signal, a motion sensor signal or a motion sensor signal of a movement of an anatomical structure of the upper airway. It is understood that one of the respective sensors required for the respective patient signals described above can be connected or is connected to the third signal input. The sensor or sensors can be an integrated part of the diagnostic and control system or can be connected separately, for example via a wire connection or wirelessly.

Preferably, the third patient signal is a dynamic pressure signal, wherein the third detector is configured to determine the severity signal as a function of a maximum value of a dynamic pressure within a first short period of time prior to the occurrence of the respiratory event signal in relation to the maximum value of a dynamic pressure within a second longer period of time prior to the occurrence of the respiratory event signal. Particularly preferably, the first short time period is 10 seconds, and the second longer time period is 60 seconds.

Preferably, the third patient signal is a pulse oximeter signal, wherein the third detector is adapted to determine the severity signal as a function of a current oxygen saturation or as a function of a statistical description of the course of the oxygen saturation in a certain period of time before the occurrence of the respiratory event signal. Particularly preferably, the third detector is designed to determine the severity signal as a function of the arithmetic mean value of the oxygen saturation in the 60 seconds prior to the occurrence of the respiratory event signal.

Alternatively, preferably, the third patient signal is an acoustic snoring sound signal, wherein the third detector is adapted to determine the severity signal from an amplitude ratio of a plurality of differently filtered partial spectra of the snoring sound signal within a first short time period prior to the occurrence of the respiratory event signal in relation to the amplitude ratio of the partial spectra within a second longer time period prior to the occurrence of the respiratory event signal. Particularly preferably, the first short time period starts 10 seconds before the occurrence of the respiratory event signal and ends directly with the occurrence of the event signal. Preferably, the second longer period begins 60 seconds before the occurrence of the respiratory event signal and ends 10 seconds before the occurrence of the respiratory event signal. Particularly preferred are the partial spectra Mel Frequency Cepstral Coefficients (MFCCs) of the snoring sound signal.

Alternatively preferred, the third patient signal is an acoustic snoring sound signal, wherein the third detector is adapted to determine the severity signal from a duration for which the amplitude of the acoustic snoring sound signal indicates a respiratory event, wherein the respiratory event is detected by measuring the decrease below a lower threshold or the increase above an upper threshold.

Preferably, the control unit processes the severity signal in such a way that the intensity of the control signal is dependent on the severity signal. Preferably, a stronger control signal is generated when the severity signal increases. A stronger control signal can then generate a stronger therapeutic effect via the therapy apparatus, such as stronger stimulation and/or longer stimulation. For example, the amplitude of the control signal may be proportional to the severity signal.

Preferably, the third detector processes the third patient signal so as to distinguish between a predetermined patient signal corresponding to hypopnoea and a predetermined other patient signal corresponding to apnoea and to generate a correspondingly different severity signal. The control unit preferably generates a different control signal when receiving the severity signal corresponding to apnoea than when receiving the severity signal corresponding to hypopnoea.

Preferably, the second detector is designed to recognize the first obstruction type and at least one second obstruction type as the most probable obstruction type from the group of predetermined obstruction types, depending on the signal at the second signal input, and to output a correspondingly indexed obstruction type signal with the first or the second obstruction type. The control unit is designed to generate the control signal when the first obstruction type is present in the obstruction type signal, and to generate a further control signal when the second obstruction type is present in the obstruction type signal, which control signal is intended to control a further therapy apparatus. For the sake of clarity, it should be stated that it is also conceivable to transmit the control signal and the further control signal to one and the same therapy apparatus, wherein the therapy apparatus is designed to perform a first type of stimulation when the control signal, which could also be referred to as the first control signal, is present, and to perform a further type of stimulation when the further control signal is present. For example, the first type of stimulation can be a functional electrical stimulation (FES) of the hypoglossal nerve and the further type of stimulation can be a functional electrical stimulation of the glossopharyngeal nerve.

Preferably, the first signal input is coupled to a microphone or microphone array, and the first detector is adapted to evaluate snoring sounds of the patient in order to form the event signal. Preferably, the second signal input is coupled to the microphone or microphone array, and the second detector is adapted to evaluate the snoring sounds of the patient in order to form the obstruction type signal.

Preferably, the first signal input is connected to a motion sensor for measuring a body movement or to a respiratory flow sensor or is connected simultaneously to the motion sensor and to the respiratory flow sensor, with the first signal input receiving two signals simultaneously and forwarding them to the first detector. The first detector processes these two signals simultaneously or together or evaluates these two signals together.

Preferably, the second detector is adapted to recognize the first obstruction type or at least one second obstruction type as the most probable obstruction type from the group of predetermined obstruction types, depending on the signal at the second signal input, and to output a corresponding obstruction type signal with the first or the second obstruction type. The control unit is adapted to generate a control signal of a first variable or intensity if the first obstruction type is present in the obstruction type signal, and to generate a control signal of a second variable or second intensity if the second obstruction type is present in the obstruction type signal.

Preferably, the control unit contains a table in which a first variable is stored for a first obstruction type and at least one second variable is stored for a second obstruction type, and the control signal variable is selected by selecting from this table. Preferably, the variable of the control signal is a voltage magnitude, a current magnitude, a duration, an edge steepness, a frequency or a duty cycle of the control signal. Preferably, each of the predefined detectable obstruction types in the table can be changed, amplified or reduced in amplitude or intensity by the control unit, where, for example, a respective amplitude has an adjustable amplification factor and/or an adjustable offset.

Preferably, the control unit is configured such that an amplitude or strength of the control signal is dependent on a stored amplification parameter, which is adaptively set and stored by the control unit depending on success or failure after the control signal has been output. Success is deemed to have occurred if the control signal output leads to a sufficient reduction in the event signal and/or the severity signal. If the event signal and/or the severity signal are not received sufficiently or reduced below a threshold after the control signal has been output, the gain parameter is increased in steps.

Preferably, the control unit has, in addition to the control output, a second control output which is intended to be coupled to another therapy apparatus or another therapy function, the control unit being designed to alternately activate first the control output and then the second control output, the control output being activated first and a first success being determined immediately or with a delay by a decaying event signal or severity signal on the basis of the first and/or the second signal input. Then the second control output is activated, and a second success is determined immediately or with a delay by a decaying event signal or severity signal using the first and/or the second signal input. A decision maker will then determine, using a comparison, whether activation of the control output or the second control output has led to greater success. The control output whose success was greater will then be enabled for activation and for evaluation of future respiratory event signals.

Preferably, the control unit is designed as a learning system that outputs the control signal in a first intensity and a second intensity that differs from the first and uses at least one of the patient signals to determine which intensity of the control signal produces the greater or equal success, and which then outputs the control signal with the respective intensity for future respiratory events.

Preferably, the control unit is designed as a learning system that controls at least one first and one second type of therapy via at least one control signal and uses at least one of the patient signals to determine which control signal is more successful in terms of the corresponding first and second type of therapy, and which then outputs the control signal which leads to highest success.

Preferably, the second detector is adapted, when determining the obstruction type signal, to evaluate the first and/or the at least one second patient signal within a predetermined time period preceding the respiratory event signal in order to determine over this preceding time period the obstruction type signal at the time of the respiratory event. For example, the obstruction type signal can be determined much better by means of tongue movements that took place 60 seconds before the respiratory event and event signal than from a signal curve of the respective patient signal at a point in time after the respiratory event. For example, the obstruction type signal can be determined much better from respiratory sound characteristics, such as snoring sounds from several snoring events, which were measured, for example, within a period of 60 seconds before the respiratory event, than from a signal curve of the respective patient signal immediately before, during or after the respiratory event. In other words, the obstruction type for a respiratory event can be determined from signal values evaluated by the second detector before, during and after the respiratory event.

Preferably, the second detector is designed as a learning system that learns from the respective patient signals to determine whether a respiratory event will occur with at least a predetermined probability within a short subsequent period of time.

Preferably, the second detector is designed as a learning system which learns from the respective patient signals to determine which obstruction type will be the most probable in a subsequent respiratory event.

A diagnostic, control and therapy system according to the invention comprises the diagnostic system as described above, and a therapy apparatus adapted to receive the control signal of the diagnostic and control system and to generate a corresponding stimulus or stimulation signal which can be transmitted to the patient to reduce an obstruction severity of the airways in the first obstruction type.

Preferably, the therapy apparatus is one of the following: a functional electrical stimulation device, an electromagnet, a controllable mandibular prognathism splint, a sleeping pillow that produces controllable deformations, a sound generator, or a vibrator.

Preferably, the functional electrical stimulation device has a pair of electrodes or several electrodes, which can be attached to or implanted in one or more muscles or nerves in the upper airways.

Preferably, the first and second detectors are designed such that they continuously process the respective input signal of the first and second signal inputs and thereby continuously determine the respective event signal and the obstruction type signal and forward them to the control unit. The control unit is designed in such a way that it continuously determines and outputs the control signal. For the sake of clarity, it shall be explained that “continuous” means that a corresponding signal processing and determination takes place repetitively, as is common in a digital processor system.

Preferred embodiments according to the present invention are shown in the following drawings and in a detailed description, but they are not intended to limit the present invention exclusively thereto.

In the following Figures:

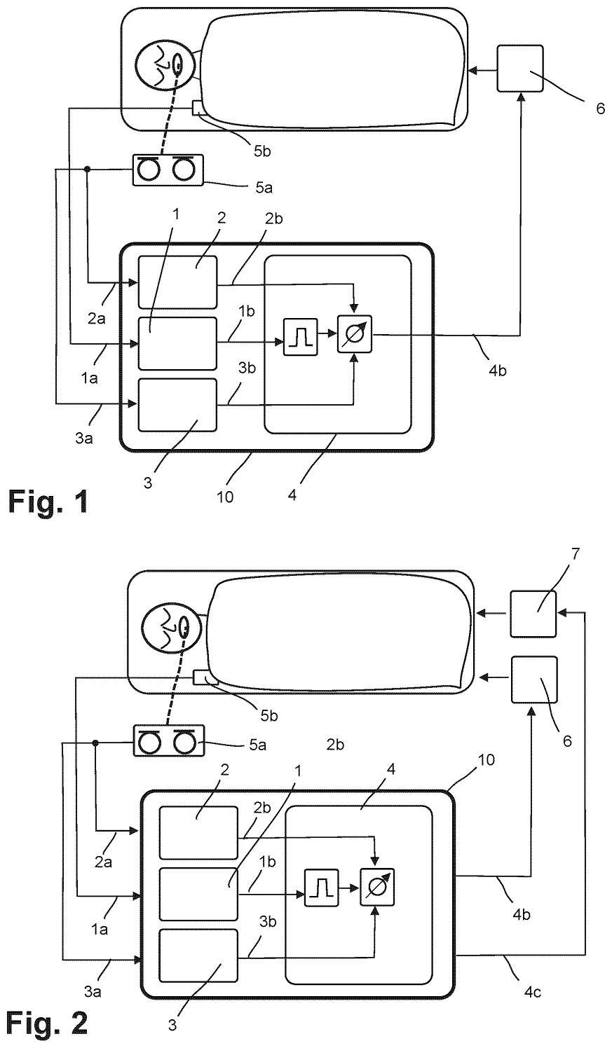

FIG. 1 shows a sketch of a sleeping patient with a preferred first embodiment of a diagnostic and control system having a first, a second and a third signal input to which respective patient signals derived from a first and a second sensor on the patient are connected, wherein in the diagnostic and control system a first, a second and a third detector process the respective patient signals to form a respective respiratory event signal, an obstruction type signal and a severity signal and feed them to a control unit, which generates a corresponding control signal that controls a therapy apparatus, which in turn sends stimulation signals to the patient.

FIG. 2 shows a sketch of the sleeping patient with a preferred second embodiment of the diagnostic and control system, which is designed like the one in FIG. 1 with the difference that the control system generates the control signal and a further control signal, where the further control signal controls a further therapy apparatus which sends different stimulation signals to the patient than the therapy device.

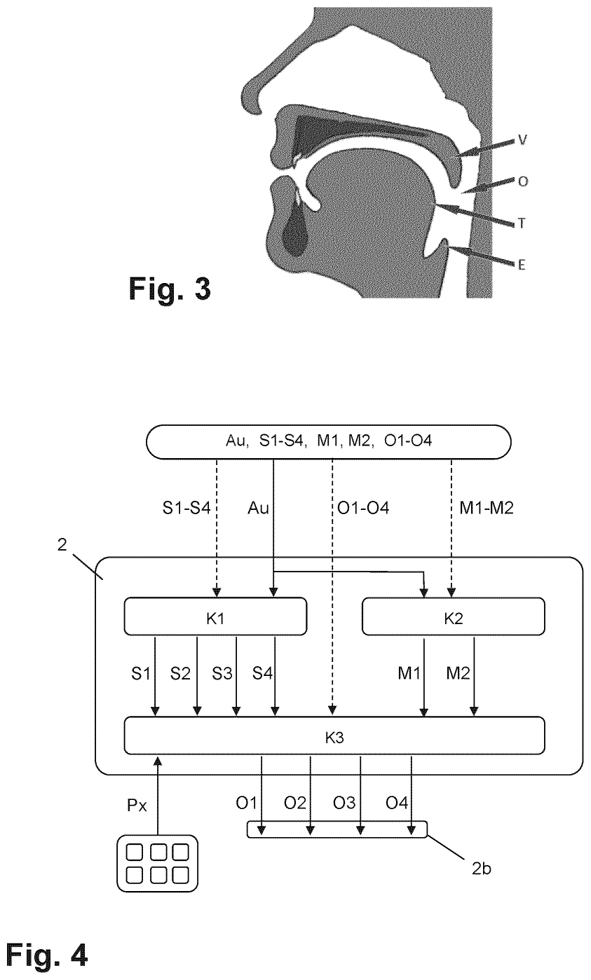

FIG. 3 shows a sectional view of a lateral view of a patient's head showing the neck, nose and throat with the areas of the velopharynx, oropharynx, base of the tongue and epiglottis:

FIG. 4 schematically shows a classification system comprising a first and a second classifier, each of which is fed a snoring sound signal with a respective optional indicator via an input interface, the output signals of the first and the second classifier being fed to a third classifier for classification, the output signals of the third classifier representing obstruction types being output via an output interface: additional snoring or patient data can be entered via an input interface and fed to the third classifier for classification; and

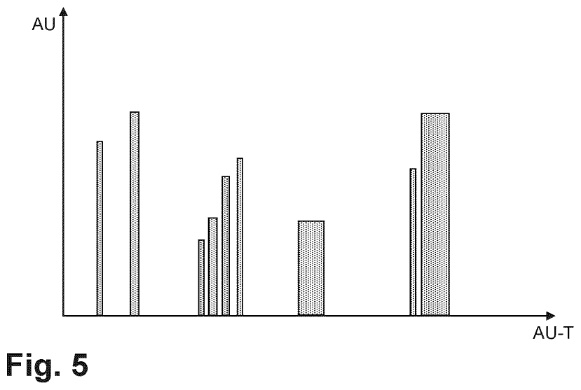

FIG. 5 shows a diagram of respiratory events along a time axis and their severity levels plotted as an amplitude.

### DETAILED DESCRIPTION OF EMBODIMENTS

The present invention relates to a diagnostic and control system 10 for continuous detection of respiratory events and their respective causative obstruction types O1-O4 of airways of a sleeping patient and for a counteracting control of a therapy apparatus 6.

In general, the diagnostic and control system 10 comprises the following:
a) a first detector 1, which receives at least one first patient signal as an electrical signal via a first signal input (1a) and which is designed to recognize when the first patient signal exceeds or falls below a predetermined tolerance range of signal values, and to generate a respiratory event signal 1b therefrom, which indicates a respiratory disorder. The predetermined tolerance range of signal values is representative of sleep without a respiratory event and is stored in the first detector (1), for example together with an average value.
b) a second detector 2, which receives the first and/or at least one second patient signal as an electrical signal via a second signal input 2a and processes it as a classifier so as to recognize a first obstruction type O1-O4 as a most probable obstruction type from a group of predetermined obstruction types O1-O4, and to output a corresponding obstruction type signal 2b with the first obstruction type O1-O4. For clarity, the group of predetermined obstruction types O1-O4 may comprise only a single first obstruction type O1, wherein the second detector recognizes whether or not this first obstruction type O1 is present with a predetermined probability and outputs it in the obstruction type signal 2b. Alternatively, the group of predetermined obstruction types O1-O4 comprises a first obstruction type O1 and a second obstruction type O2, wherein the second detector recognizes which of the obstruction types is likely to be present with more than the predetermined probability and outputs it in the obstruction type signal 2b. Similarly, the group of predetermined obstruction types O1-O4 may comprise three, four or more obstruction types O1-O4, wherein the second detector recognizes which of the obstruction types O1-O4 is present with a predetermined probability, and outputs it in the obstruction type signal 2b.
c) a control unit 4 which receives the event signal 1b and the obstruction type signal 2b and, if the event signal 1b is positive and the obstruction type signal 2b corresponds to the first obstruction type O1-O4, generates a control signal 4b which can be used to control the therapy apparatus 6.

FIG. 1 shows a preferred first embodiment of the diagnostic and control system, wherein the first patient signal 1a comes from a second sensor 5b, which is, for example, a respiratory flow sensor. In this case, the respiratory event signal is derived from a respiratory flow signal, for example when a respiratory flow value falls below a threshold value in the first detector 1. The second patient signal 2a comes from a first sensor 5a, which is a microphone array that picks up snoring noises and converts them into an electrical signal. From the snoring sound signal, the second detector 2 and its classifier detect the most probable obstruction type, which enables the control signal 4b, for example, when the first obstruction type has been detected. In the preferred embodiment of the diagnostic and control system, a third detector 3 is also implemented, which detects a third patient signal 3a, which in this case is preferably the same as the second patient signal, which is the snoring noise signal. The third detector 3 forms a severity signal from the third patient signal 3a, which represents the severity of the respiratory event. Similarly, the third patient signal 3a could also be used by a pulse oximeter, as the severity of a respiratory event is also linked to the SpO2 value of the patient's blood. For example, the severity signal 3b could modulate the control signal 4b when the respiratory event signal 1b and the obstruction type signal 2b occur, so that with an increasing severity signal the control signal 4b would also be made more intense, for example by a higher amplitude or frequency. The control signal 4b is used to control the therapy apparatus 6, which stimulates a part of the patient, such as a hypoglossal nerve, by means of functional electrical stimulation.

A preferred other embodiment of the diagnostic and control system provides a control unit 4, which can generate the control signal 4b and a further control signal 4c, which controls a further therapy apparatus 7. The additional therapy apparatus 7 is designed to perform a different type of stimulation on the patient than the therapy apparatus, or the first therapy apparatus, respectively. The additional therapy apparatus 7 can either stimulate a different nerve or perform a completely different type of stimulation, such as stimulation to change the body position using a sleep position trainer.

FIG. 3 shows a sectional view of a side view of a patient's head, showing the neck, nose and throat with the areas velopharynx (plane of the soft palate) V, oropharynx (plane of the palatine tonsils) O, plane of the base of the tongue T and epiglottis E.

FIG. 4 schematically shows a preferred classification system with a first classifier K1 and a second classifier K2, to each of which a snoring sound signal Au with a respective optional indicator (S1-S4) is fed via an input interface, with first output signals S1-S4 of the first classifier K1 and second output signals M1-M2 of the second classifier K2 being fed to a third classifier K3 for classification. The third classifier K3 recognizes the obstruction type O1-O4 from the first output signals S1-S4 and the second output signals M1-M2 and maps the former in its output signals O1-O4, which together produce the obstruction type signal 2b. The classification system can learn with the aid and input of known obstruction types O1-O4 and mouth positions M1-M2 in conjunction with the snoring sound signal. Additional patient data Px can also be entered via an input interface for learning or subsequent classification. Such a classification system for analyzing the snoring signal Au is disclosed, for example, in DE 1020 19 105 762 A1.

FIG. 5 shows a diagram of respiratory events as snoring sound signals AU along a time axis AU-T, wherein the respective amplitudes of the snoring sound signals AU can also be evaluated as degrees of severity. A groupwise increasing degree of severity can be recognized, according to which the respective control signal was triggered, which is not shown, and which leads to a respective interruption of the respective respiratory events until they start again.

A method is also presented for a diagnostic and a control system which recognizes respiratory events and their at least one causative obstruction type O1-O4 of the airways of the sleeping patient, and which thereby generates a control signal for the therapy apparatus 6; comprising the following steps:
a) receiving, by a first detector 1, of a first patient signal from the patient in which a respiratory event is recognizable, and detecting of the respiratory event when the first patient signal exceeds or falls below a predetermined tolerance range, whereby a respiratory event signal 1b is generated which is representative of a respiratory disorder, the predetermined tolerance range being representative of sleeping without a respiratory event;
b) by a second detector 2, recognizing at least one first obstruction type O1-O4 as a most probable obstruction type from a group of predetermined obstruction types O1-O4 in the first patient signal and/or in at least one second patient signal, and generating a corresponding obstruction type signal 2b with the first obstruction type O1-O4; and
c) by a control unit 4, receiving and detecting the event signal 1b and the obstruction type signal 2b corresponding to the first obstruction type O1-O4, and thereby generating the control signal 4b for a corresponding control of the therapy apparatus 6 which counteracts the first obstruction type.

The first and second patient signals are each a signal from one or more sensors, as described above.

A preferred further step is a receiving and processing of a third patient signal 3a, which is the first and/or the second and/or another third patient signal of a sensor on/near the patient, from an amplitude or a time response, to form a severity signal 3b, which is a function of a severity of the respiratory event, wherein the control unit 4 processes the severity signal 3b such that the control signal 4b is a function of the severity signal 3b.

Preferably, both the first obstruction type and a second obstruction type can be recognized from the first and/or the second patient signal by the second detector, with the first control signal 4b being generated when the first obstruction type is present and a further control signal 4c being generated when the second obstruction type is present. The further control signal 4c is preferably used to control a further therapy apparatus. Alternatively, the further control signal 4c is preferably the control signal with a different magnitude or intensity, so that the therapy apparatus 6 is controlled differently when the first type of obstruction is present than when the second type of obstruction is present.

Preferably, an amplitude or intensity of the control signal (4b) is generated depending on a stored amplification parameter, which is preferably set and stored adaptively by the control unit 4 depending on success or failure after the output of the control signal 4b. It is considered a success if the output control signal 4b leads to a sufficiently large reduction in the event signal 1b and/or the severity signal 3b. Preferably, if the event signal 1b and/or the severity signal 3b are not received to be sufficiently reduced after the control signal 4b is output, the gain parameter is increased in steps. Preferably, the control signal 4b is increased or amplified stepwise until the event signal 1b and/or the severity signal 3b are received to be sufficiently reduced.

Further method steps can also be taken from the above description.

For the sake of clarity, it should be noted that indefinite articles in connection with an object, or numerical indications, for example indicating “one” object, do not limit the object numerically to exactly one object, but are intended to indicate a minimum of “one” object. This applies to all indefinite articles such as “a”, “an” etc.

It is understood that when an item is described as being “on”, “attached to”, “coupled to” or “in contact with” another item, the item may then be directly on, attached to or coupled to the other item, or there may also be intervening items which are either merely interposed or connect or couple or keep in contact the item with the other item. On the other hand, when an element is described as being “directly on” another element, “directly connected”, “directly coupled” or “directly in contact” with it, it is to be understood that there are no intervening elements. Similarly, when a first element is referred to as being “in electrical contact with” or “electrically coupled to” a second element, an electrical pathway is present that allows current to flow between the first element and the second element. The electrical path may include capacitors, coupled inductors and/or other elements that allow current to flow even without direct contact between the conductive elements.

Although the terms “first”, “second”, etc. may be used herein to refer to various elements, components, regions and/or sections, such elements, components, regions and/or sections are not limited by these terms. The expressions are used only to distinguish one element, one component, region or section from another element, another component, region or section. Therefore, a first element, component, region or section discussed below may be referred to as a second element, component, region or section without departing from the teachings of the present invention.

Concerning the term “comprising”, it should be said for clarity that when a first device part comprises a second device part, this means that the first device part “comprises” the second device part and does not necessarily enclose it in terms of arrangement, unless it is, for example, a description of a positional and shape arrangement; the same applies to a method, which may comprise one or more method steps.

Further possible embodiments are described in the following claims. In particular, the various features of the embodiments described above can also be combined with one another, provided they are not technically mutually exclusive.

The reference numbers mentioned in the claims are only for better comprehensibility and do not limit the claims in any way to the embodiments shown in the figures.

### LIST OF REFERENCE NUMBERS

1 first detector
1a first signal input
1b event signal
2 second detector
2a second signal input
2b obstruction type signal
3 third detector
3a third signal input
3b severity signal
4 control unit
4b control signal
4c additional control signal
5a first sensor
5b second sensor
6 therapy apparatus
7 additional therapy apparatus
10 diagnostic system
AU snoring sound signal
AU-T time axis of snoring sound signal
S1-S4 snoring sound origin type
M1, M2 mouth position
O1-O4 obstruction type
K1 first classifier
K2 second classifier
K3 third classifier
Px snoring or patient data
V velopharynx
O oropharynx
T area of base of tongue
E epiglottis area

1. A diagnostic and control system for continuous detection of respiratory events and their respective causative types of obstruction of the airway of a sleeping patient and for counteracting control of a therapy apparatus, comprising:
a) a first detector which receives at least one first patient signal as an electrical signal via a first signal input and is designed to recognize when the first patient signal exceeds or falls below a predetermined tolerance range of signal values and to generate a respiratory event signal therefrom which is representative of a respiratory disorder, wherein the predetermined tolerance range of the signal values is representative of sleeping without a respiratory event and is stored in the first detector;
b) a second detector which receives the first and/or at least one second patient signal as an electrical signal via a second signal input and processes it as a classifier so as to recognize therefrom a first obstruction type as a most probable obstruction type from a group of predetermined obstruction types and to output a corresponding obstruction type signal with the first obstruction type; and
c) a control unit which is adapted to receive the event signal and the obstruction type signal and to detect the event signal as a positively existing signal and the obstruction type signal with the first obstruction type, generating therefrom a control signal which can be used to control the therapy apparatus.

2. The diagnostic and control system according to claim 1, wherein the first patient signal is one or a combination of the following: a pulse oximeter signal, a microphone signal of a breathing and/or snoring sound, a respiratory flow signal, an air pressure signal, a chest muscle signal or a breathing belt signal, an EEG signal, an ECG signal, an EMG signal, a position sensor signal, a motion sensor signal or a motion sensor signal of a movement of an anatomical structure of the upper airway.

3. The diagnostic and control system according to claim 1, wherein the second patient signal is one or a combination of the following: the first patient signal, a microphone signal of a breathing and/or snoring sound, a respiratory flow signal, a signal of a palate sensor, a signal of a tongue position sensor, an air pressure sensor signal, a combination of several air pressure sensor signals, an EMG signal or a movement sensor signal of a movement of an anatomical structure of the upper airway.

4. The diagnostic and control system according to claim 1, comprising a third detector which is supplied with a third patient signal which is the first and/or the second and/or another third patient signal of a sensor on or near the patient, and which the third detector evaluates with respect to an amplitude or a time response such that it thereby generates a severity signal which is a function of a severity of the respiratory event, wherein the control unit receives the severity signal and processes it such that the control signal is a function of the severity signal.

5. The diagnostic and control system according to claim 1, the second detector being adapted to recognize, depending on the signal at the second signal input, each of the first obstruction type and at least one second obstruction type as the most probable obstruction type from the group of predetermined obstruction types, and to output a corresponding obstruction type signal with the respective first or second obstruction type; the control unit being adapted to generate the control signal if the first obstruction type is present in the obstruction type signal and to generate, if the second obstruction type is present in the obstruction type signal, an additional control signal which is provided for controlling an additional therapy apparatus.

6. The diagnostic and control system according to claim 1, the second detector being adapted to recognize, depending on the signal at the second signal input, the first obstruction type or at least one second obstruction type as the most probable obstruction type from the group of predetermined obstruction types, and to output a corresponding obstruction type signal with the first or the second obstruction type,
the control unit being adapted to generate, if the first obstruction type is present in the obstruction type signal, a control signal of a first variable, and, if the second obstruction type is present in the obstruction type signal, to generate a control signal of a second variable.

7. The diagnostic and control system according to claim 1, the control unit being adapted so that an amplitude or intensity of the control signal is generated in dependence on a stored amplification parameter, which is set and stored adaptively by the control unit depending on success or failure after the output of the control signal, a reduction in the event signal or the severity signal being determined over a time period, where it is considered a success if the output control signal leads to a predetermined sufficiently large reduction in the event signal and/or the severity signal, and where, if no sufficient reduction in the event signal and/or the severity signal are detected after the control signal is output, the gain parameter is increased in steps.

8. The diagnostic and control system according to claim 1, where the control unit has, in addition to the control output, a second control output which is intended to be coupled to another therapy apparatus or another therapy function, the control unit being designed to alternately activate first the control output and then the second control output, with a reduction of the event signal or the severity signal being determined over time, the control output being activated first and, using the first and/or the second signal input, a first success being determined with a delay by a reduction of the event signal or severity signal; and then the second control output being activated and a second success being determined with a delay by a reduction of the event signal or severity signal using the first and/or the second signal input; whereupon a decision maker will then determine, using a comparison of the first reduction with the second reduction, whether activation of the control output or the second control output has led to greater success, whereupon the control output whose success was greater will then be enabled for activation and for future respiratory event signals.

9. The diagnostic and control system according to claim 1, the second detector being adapted to evaluate, for determining the obstruction type signal, the first and/or the at least one second patient signal within a predetermined time period preceding the respiratory event signal in order to determine over this preceding time period the obstruction type signal at the time of the respiratory event.

10. The diagnostic, control and therapy system consisting of a diagnostic system according to claim 1 and a therapy apparatus which is adapted to receive the control signal of the diagnostic and control system and to generate a corresponding stimulus for the patient which is supplied to the patient and which reduces, in case of a first obstruction type of a group of predetermined obstruction types, an obstruction severity of the airway.

11. The diagnostic, control and therapy system according to claim 10, wherein the therapy apparatus is one of the following: a functional electrical stimulation device, an electromagnet, a controllable mandibular prognathism splint, a sleeping pillow that produces controllable deformations, a sound generator, or a vibrator.
